(19) United States
(12) Patent Application Publication
(10) Pub. No.: US 2026/0335074 A1
(43) Pub. Date: Oct. 29, 2026

(54) SYSTEMS, DEVICES, AND METHODS FOR TREATING HEART VALVES

(71) Applicant: Edwards Lifesciences Corporation, Irvine, CA (US)

(72) Inventors: Kevin Gantz, Laguna Beach, CA (US); Sean Chow, Irvine, CA (US); Ronaldo C. Cayabyab, Mission Viejo, CA (US); Darshin S. Patel, Lake Forest, CA (US); Tri D. Tran, Fountain Valley, CA (US)

(21) Appl. No.: 19/698,335

(22) Filed: Jun. 4, 2026

Related U.S. Application Data
(63) Continuation of application No. PCT/US2024/058815, filed on Dec. 6, 2024.
(60) Provisional application No. 63/610,991, filed on Dec. 15, 2023.

(51) Int. Cl. A61F 2/24

(52) U.S. Cl. CPC A61F 2/2418; A61F 2/2433; A61F 2220/0025; A61F 2230/0091; A61F 2250/006

(57) ABSTRACT

An assembly can include a docking device and a stabilization device configured to maintain the docking device at a selected implantation location during an implantation procedure. The docking device can include a coil extending along a central axis, the coil having a central region including a plurality of turns and a leading turn extending from a distal end portion of the central region. The stabilization device can be releasably coupled to a proximal end portion of the docking device, and can include a wire and a hypotube member having a first portion comprising a spiral cut and a second portion comprising a plurality of slots.

### CROSS REFERENCE TO RELATED APPLICATION

This application is a continuation of PCT Patent Application No. PCT/US2024/058815, filed on Dec. 6, 2024, with claims priority to and the benefit of U.S. Provisional Application No. 63/610,991, filed Dec. 15, 2023, the entire disclosure of each of these applications being incorporated herein by this reference.

### FIELD

The present disclosure relates to delivery apparatuses for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (e.g., through a femoral artery or femoral vein) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

In some examples, a docking device can be implanted first within the native valve and can be configured to receive a prosthetic valve and secure (e.g., anchor) the prosthetic valve in a desired position within the native valve. For example, the docking device can form a more circular and/or stable anchoring site at the native valve annulus in which a prosthetic valve can be expanded and implanted. A transcatheter delivery apparatus can be used to deliver the docking device to the implantation site.

### SUMMARY

Described herein are prosthetic heart valves, docking devices, delivery apparatus, and methods for implanting docking devices and prosthetic heart valves. The disclosed prosthetic heart valves, docking devices, delivery apparatus, and methods can, for example, help maintain an implanted docking device at a selected location during a prosthetic heart valve implantation procedure. As such, the devices and methods disclosed herein can, among other things, overcome one or more of the deficiencies of some typical prosthetic heart valves, docking devices, and their delivery apparatus.

A delivery system for implanting one or more prosthetic implants can generally comprise a guide catheter, and one or more prosthetic implant delivery apparatuses. In addition to these components, a delivery system can further comprise one or more of the components disclosed herein.

A guide catheter can comprise a handle and one or more shafts coupled to the handle.

A prosthetic implant delivery apparatus can comprise a handle and one or more shafts coupled to the handle.

In some examples a prosthetic implant can be a prosthetic heart valve. In some examples a prosthetic heart valve can comprise a frame and a valvular structure coupled to the frame.

In some examples a prosthetic implant can be a docking device. In some examples a docking device can comprise a coil and one or more turn portions.

In some examples, the docking device can comprise a proximal nut configured to couple a stabilization device.

In some examples, a delivery system can further comprise a stabilization device configured to maintain a docking device at a selected implantation location during an implantation procedure.

In some examples, the stabilization device can comprise a wire and a hypotube member.

In some examples, the stabilization device can comprise a coupling member configured to releasably couple the docking device.

In some examples, the coupling member comprises a threaded engagement portion configured to couple a correspondingly threaded inner bore on a proximal nut of the docking device.

In some examples, the proximal nut further comprises one or more wing members extending radially from a main body of the proximal nut.

In some examples, the hypotube member has a first portion comprising a spiral cut and a second portion comprising a plurality of slots.

In some examples, a stabilization device can comprise one or more of the components recited in Examples 1-20 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a stage in an example mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the example mitral valve replacement procedure where a docking device delivery apparatus extending through the guide catheter is implanting a docking device for a prosthetic heart valve at the native mitral valve.

FIG. 2B schematically illustrates another stage in the example mitral valve replacement procedure where the docking device of FIG. 2A is fully implanted at the native mitral valve of the patient and the docking device delivery apparatus has been removed from the patient.

FIG. 3A schematically illustrates another stage in the example mitral valve replacement procedure where a prosthetic heart valve delivery apparatus extending through the guide catheter is implanting a prosthetic heart valve in the implanted docking device at the native mitral valve.

FIG. 3B schematically illustrates another stage in the example mitral valve replacement procedure where the prosthetic heart valve is fully implanted within the docking device at the native mitral valve and the prosthetic heart valve delivery apparatus has been removed from the patient.

FIG. 4 schematically illustrates another stage in the example mitral valve replacement procedure where the guide catheter and the guidewire have been removed from the patient.

FIG. 5 is side view of an exemplary guide catheter configured to receive a delivery apparatus and guide the delivery apparatus through a portion of a patient’s vasculature, according to an example.

FIG. 6 is a side view of a delivery apparatus for a docking device, according to an example.

FIG. 7 is a perspective view of a docking device, according to an example.

FIG. 8 is a perspective view of a delivery apparatus for a prosthetic heart valve, according to an example.

FIG. 9 is a perspective view of a prosthetic heart valve, according to an example.

FIG. 10 is a perspective view of docking device, according to an example.

FIG. 11 is a perspective view of the docking device of FIG. 10, including a guard member.

FIG. 12 is a perspective view of a proximal nut of the docking device of FIG. 10, according to one example.

FIG. 13 is a perspective view a stabilizing device, according to one example.

FIG. 14 is a side view of a hypotube member of the stabilizing device, according to one example.

FIG. 15 is a perspective view of a portion of the hypotube member of the stabilizing device of FIG. 13.

FIG. 16 is a perspective view of a portion of the hypotube member of the stabilizing device of FIG. 13.

FIG. 17 is a top down view of a window member of a stabilizing device, according to one example.

FIG. 18 is a perspective view of a stabilizing device, according to one example.

FIGS. 19-22 are various views of a coupling member of a stabilizing device, according to one example.

FIGS. 23-26 are various views of a proximal stopper of a stabilizing device, according to one example.

FIGS. 27-31 show an exemplary method of implanting a prosthetic heart valve within an implanted docking device coupled to a stabilization device, according to one embodiment.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient’s body), while distal motion of the device is motion of the device away from the user and toward the implantation site (e.g., into the patient’s body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

Described herein are examples of a steerable delivery apparatus (sometimes referred to as a steerable catheter) that can be used to navigate a subject’s vasculature to deliver an implantable, expandable medical device (such as a docking device or a prosthetic heart valve), tools, agents, or other therapy to a location within the body of a subject. Examples of procedures in which the steerable catheters are useful include neurological, urological, gynecological, fertility (such as in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

In connection therewith, various systems, apparatuses, methods, or the like are described herein that, in some examples, can help ensure the expandable medical device is expanded to its functional size when implanted within the body of the subject.

A defective native heart valve may be replaced with a transcatheter prosthetic heart valve in a transcatheter heart valve replacement procedure. Typically, the prosthetic heart valve can be implanted within a native valve annulus of the defective native heart valve using a delivery apparatus (which is also referred to herein as a “prosthetic heart valve delivery apparatus” and/or a “balloon catheter”). Typically, the delivery apparatus can comprise a shaft and an inflatable balloon mounted to a distal end portion of the shaft. The prosthetic heart valve can be mounted around the balloon, which can inflate to radially expand the prosthetic heart valve at the native valve annulus.

In some examples, the prosthetic heart valve may not be able to sufficiently conform to the geometry of the native tissue (for example, to the leaflets and/or annulus of the native heart valve) and may undesirably shift around relative to the native tissue, which can lead to paravalvular leakage. Thus, a docking device may be implanted first at the native valve annulus and then the prosthetic heart valve can be implanted within the docking device to help anchor the prosthetic heart valve to the native tissue and provide a seal between the native tissue and the prosthetic heart valve.

However, in some examples, the docking device may move or migrate within the native valve to an undesirable location, position, or orientation. Thus, the inventors have discovered a need for a method or device to maintain the docking device at the selected implantation location after removal of the dock delivery apparatus and prior to the implantation of the prosthetic heart valve.

Thus, to address one or more problems of known docking device and prosthetic heart valve implantation systems and methods, it would be desirable to design a stabilization device to maintain the docking device at the selected implantation location in the selected position/orientation during the implantation procedure.

Described herein are some exemplary stabilization devices that can, in some examples, help ensure that a docking device is maintained at a selected implantation location. In some examples, this can include retaining the docking device in a desired position and/or orientation.

### Examples of the Disclosed Technology

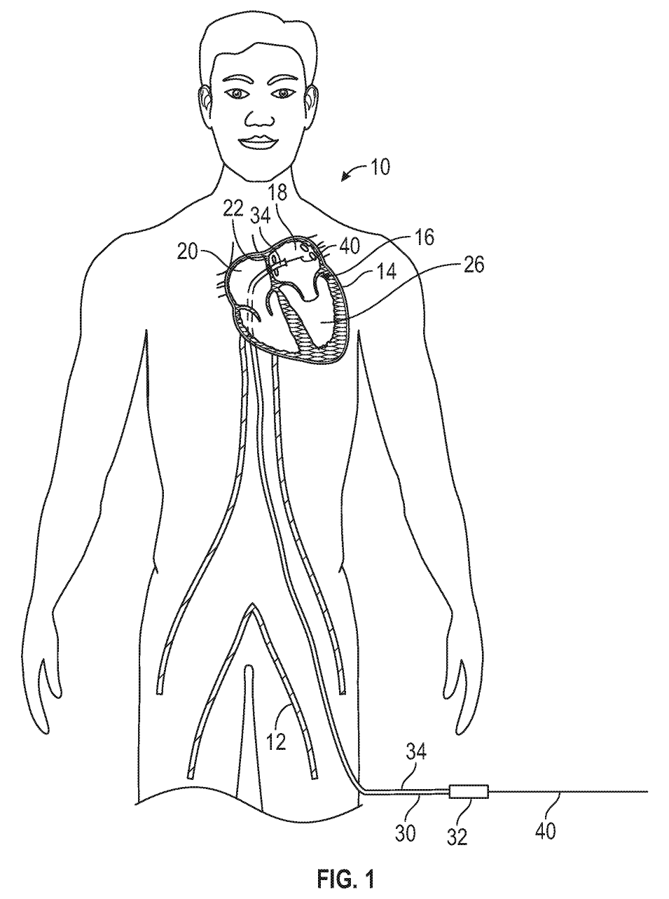

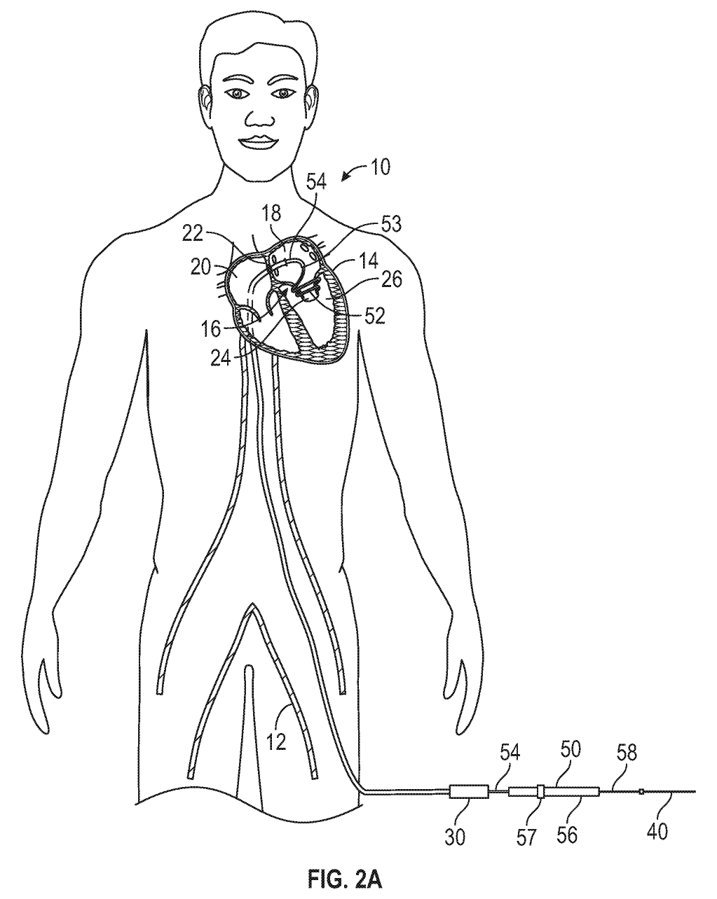

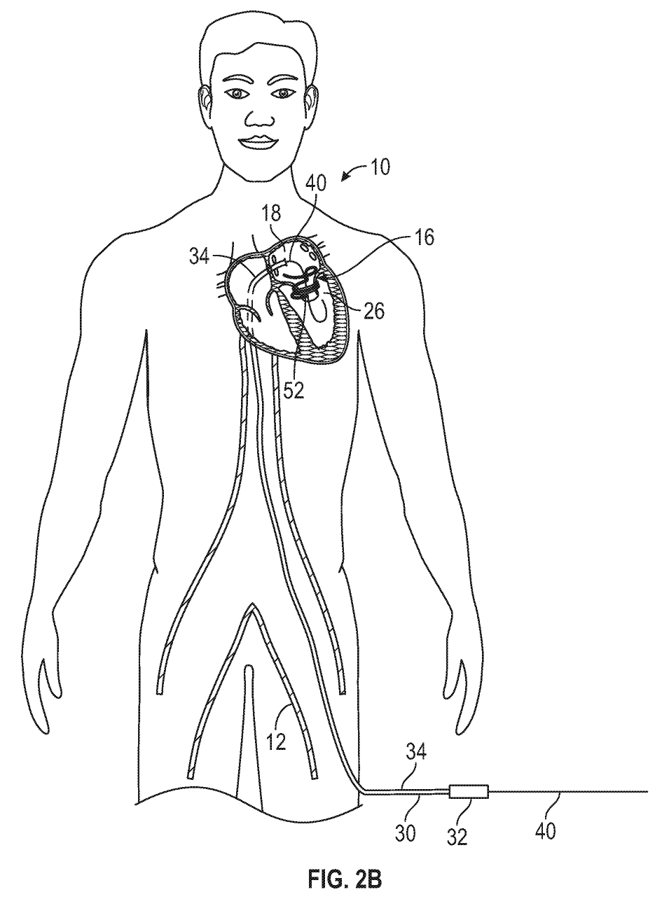

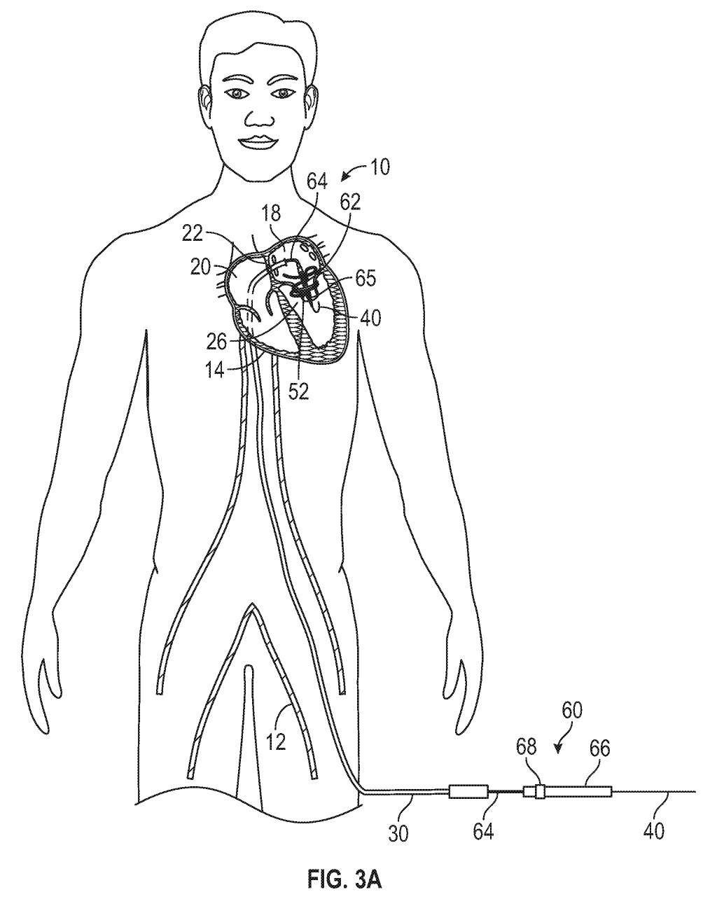

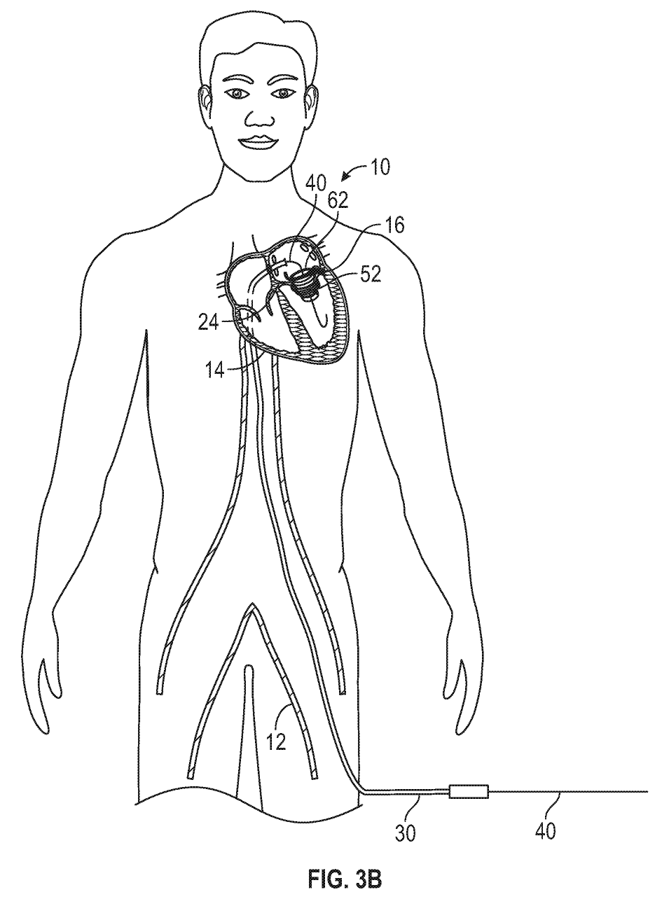

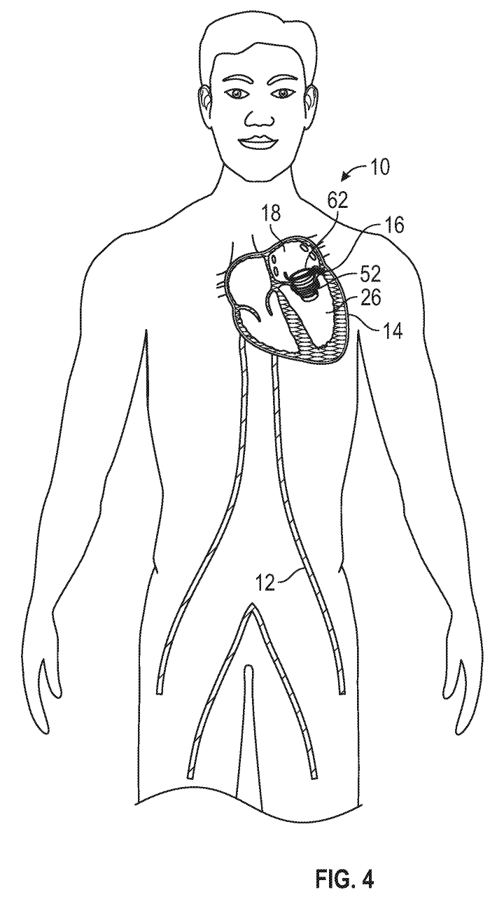

FIGS. 1-4 depict an example of a transcatheter heart valve replacement procedure (for example, a mitral valve replacement procedure) which utilizes a docking device 52 and a prosthetic heart valve 62, according to one example. During the procedure, a user first creates a pathway to a patient’s native heart valve using a guide catheter 30 (FIG. 1). The user then delivers and implants the docking device 52 at the patient’s native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (for example, the native mitral valve 16 or native mitral valve annulus). As shown, the heart 14 is illustrated schematically. For example, the anterior leaflet and chordae of the native mitral valve 16 are omitted for illustration purposes, such that only a portion of the posterior leaflet of the native mitral valve 16 is illustrated.

Initially, the user may first make an incision in the patient’s body to access the blood vessel 12. For example, in the example illustrated in FIG. 1, the user may make an incision in the patient’s groin to access a femoral vein. Thus, in such examples, the blood vessel 12 may be a femoral vein.

After making the incision at the blood vessel 12, the user may insert the guide catheter 30, the guidewire 40, and/or additional devices (for example, an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (for example, the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (for example, the guide catheter 30, the docking device delivery apparatus 50, the prosthetic valve delivery apparatus 60, additional catheters, or the like) and their associated devices (for example, docking device, prosthetic heart valve, and the like) to the implantation site within the heart 14, and thus may extend all the way through the blood vessel 12 and into a left atrium 18 of the heart 14 (FIG. 1) and in some examples, through the native mitral valve 16 and into a left ventricle of the heart 14.

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (for example, through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient’s vasculature (blood vessel 12) and to the implantation site (for example, the native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient’s vasculature (for example, the blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (for example, the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO 2020/247907, which is incorporated by reference herein in its entirety.

Referring again to FIG. 2A, after the guide catheter 30 is positioned within the left atrium 18, the user may insert the docking device delivery apparatus 50 (for example, the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (for example, pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (for example, the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (for example, the portion of the docking device 52 shown in FIG. 2A that is configured to be positioned within a left ventricle 26 and/or on the ventricular side of the native mitral valve 16), the user may then deploy the remaining portion of the docking device 52 (for example, an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient’s vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient’s vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (for example, the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, inflate the inflatable balloon), thereby radially expanding the prosthetic heart valve 62 within the docking device 52.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (for example, tricuspid, pulmonary, and/or aortic valves). Further, the same and/or similar delivery apparatuses (for example, docking device delivery apparatus 50, prosthetic valve delivery apparatus 60, guide catheter 30, and/or guidewire 40), docking devices (for example, docking device 52), replacement heart valves (for example, prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient’s vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

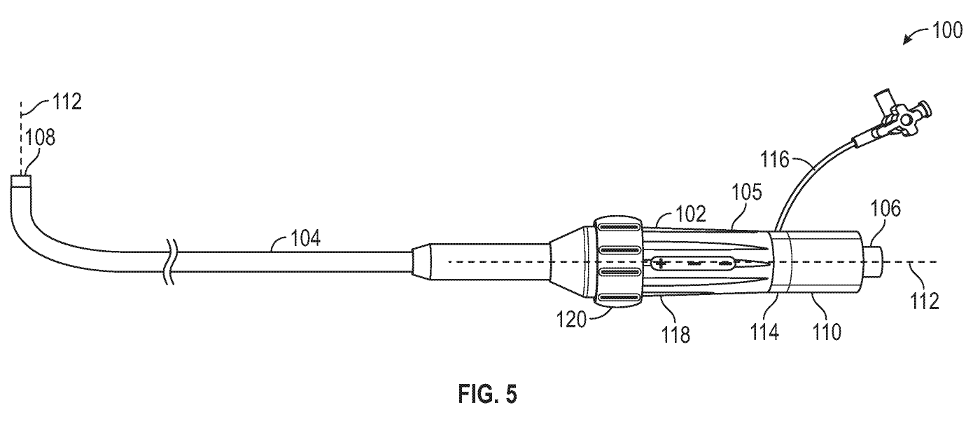

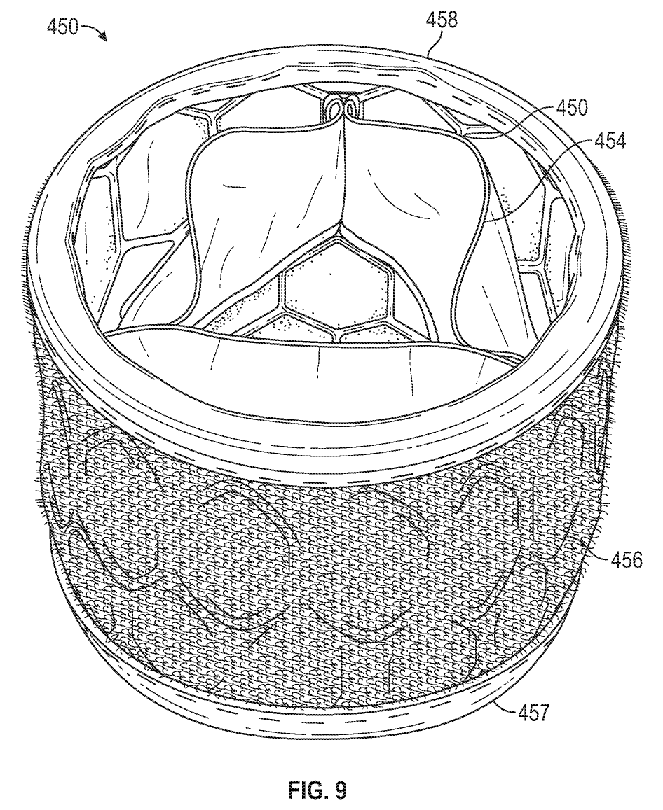

FIG. 5 illustrates an exemplary guide catheter, which is referred to below as a guide sheath 100 (and can also be referred to herein as a “delivery apparatus” or an “introducer device”). The guide sheath 100 can be configured to be inserted into a patient’s vasculature and receive a balloon catheter or delivery apparatus therein in order to introduce the balloon catheter into the patient’s vasculature and at least partially guide the balloon catheter therein to a target implantation site. For example, the guide sheath 100 can be used as the guide sheath 30 in a prosthetic valve implantation procedure, as described above with reference to FIGS. 1-4. An exemplary balloon catheter for a prosthetic medical device (referred to below as “delivery apparatus 400”) that can be received within the guide sheath 100 is shown in FIG. 9, as described further below. Though the guide sheath 100 is described herein as being used with the delivery apparatus 400, the guide sheath 100 can be configured to receive a variety of delivery apparatuses, balloon catheters, or implant catheters, such as alternate prosthetic heart valve delivery apparatuses, docking device delivery apparatuses, and/or delivery apparatuses for other prosthetic medical devices or medical therapies, such as stents.

The guide sheath 100 in the illustrated example comprises a handle 102, an elongated shaft 104 extending distally from the handle 102, and a central longitudinal axis 112. The shaft 104 has a main (or primary) lumen that is defined by an inner surface of a wall of the shaft 104. The main lumen is configured to receive a delivery apparatus therein (such as any of the prosthetic device delivery apparatuses or implant catheters described herein). In some examples, the shaft 104 can extend into the handle 102. Further, in some examples, the main lumen can extend through the handle 102 to an inlet port 106 disposed at a proximal end of the handle 102. Thus, in some examples, an inner surface of a wall of a portion of the handle (for example, at the proximal end) can further define the main lumen. Thus, the main lumen can extend from the inlet port 106 to a distal end 108 of the shaft 104.

The handle 102 can have an outer housing 105 and can further include a seal housing assembly 110 (which can also be referred to as a “seal stack”) which comprises one or more seals contained therein. The one or more seals of the seal housing assembly 110 can be configured to fluidly seal the main lumen of the guide sheath 100 from the external environment. For example, the one or more seals of the seal housing assembly 110 can be configured to prevent blood from a patient in which the guide sheath 100 is inserted from exiting the guide sheath 100 and prevent air from the environment from entering the guide sheath 100 (for example, through the inlet port 106). The one or more seals can include a variety of types of seals, such as a duckbill seal, a flapper seal, an umbrella valve, a cross-slit valve, a dome valve, or the like.

The handle 102 can, in some instances, include an adaptor spine 114 disposed adjacent and distal to the seal housing assembly 110. A flush port 116 can be connected to the outer housing 105 at the adaptor spine 114. A flush lumen of the adaptor spine 114 is connected to the flush port 116 and further connects to the main lumen. The flush port 116 can be configured to receive fluid through a lumen thereof. In this way, the flush port 116 can be fluidly coupled to the main lumen by the flush lumen.

The handle 102 can include a steering mechanism configured to adjust the curvature of the distal end portion of the shaft 104 (as such, the shaft 104 can be referred to as a steerable shaft). In the illustrated example, the handle 102 includes a main body portion 118 disposed adjacent and distal to the adaptor spine 114 and an adjustment member, such as the illustrated rotatable knob 120. The main body portion 118 can house internal flex mechanisms of the guide sheath 100 which are operatively coupled to the rotatable knob 120. In some examples, the flex mechanisms, and thus the knob 120, can be operatively coupled to the proximal end portion of a pull wire. The pull wire can extend distally from the handle 102 through the shaft 104 and have a distal end portion affixed to the shaft 104 at or near the distal end 108 of the shaft 104. Rotating the knob 120 can increase or decrease the tension in the pull wire, thereby adjusting the curvature of the distal end portion of the shaft 104. Further details on steering or flex mechanisms for a delivery apparatus can be found in U.S. Patent No. 9,339,384, which is incorporated by reference herein.

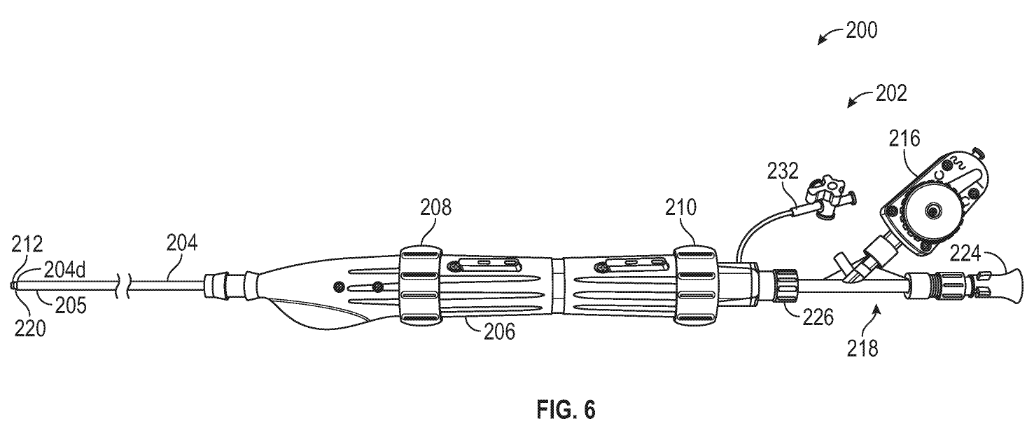

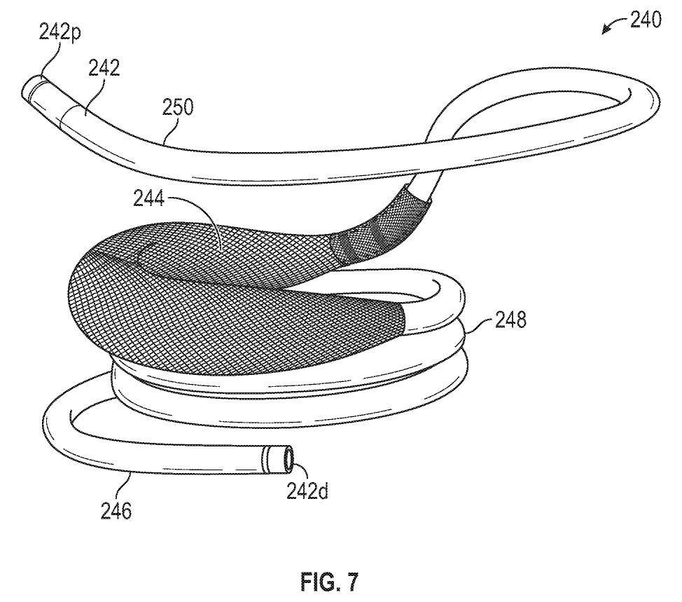

FIG. 6 illustrates a delivery apparatus 200 configured to implant a docking device, such as docking device 240 (FIG. 7) described below or other docking devices, to a target implantation site in a patient, according to one example. For example, the delivery apparatus 200 can be used as the docking device delivery apparatus 50 in a prosthetic valve implantation procedure, as described above with reference to FIG. 2A. The delivery apparatus 200 can also be referred to as a “dock delivery catheter” or “dock delivery system.”

As shown, the delivery apparatus 200 can include a handle assembly 202 and a delivery sheath 204 (also referred to as the “delivery shaft” or “outer shaft” or “outer sheath”) extending distally from the handle assembly 202. The handle assembly 202 can include a handle 206 including one or more knobs, buttons, wheels, and/or other means for controlling and/or actuating one or more components of the delivery apparatus 200. For example, in some examples, as shown in FIG. 6, the handle 206 can include knobs 208 and 210 which can be configured to steer or control flexing of the delivery apparatus 200 such as the delivery sheath 204 and/or a sleeve shaft 220 described below.

In certain examples, the delivery apparatus 200 can also include a pusher shaft 212 and a sleeve shaft 220, both of which can extend through an inner lumen of the delivery sheath 204 and have respective proximal end portions extending into the handle assembly 202.

As described below, a distal end portion (also referred to as “distal section”) of the sleeve shaft 220 can be configured to cover (for example, surround) the docking device 240 (see FIG. 7). For example, the docking device 240 can be retained inside the sleeve shaft 220, which is further retained by a distal end portion 205 of the delivery sheath 204, when navigating through a patient’s vasculature.

Additionally, the distal end portion 205 of the delivery sheath 204 can be configured to be steerable. In one example, by rotating a knob (for example, one of knobs 208 or 210) on the handle 206, a curvature of the distal end portion 205 can be adjusted so that the distal end portion 205 of the delivery sheath 204 can be oriented in a desired angle. For example, to implant the docking device 240 at the native mitral valve location, the distal end portion 205 of the delivery sheath 204 can be steered in the left atrium so that at least a portion of the sleeve shaft 220 and the docking device 240 retained therein can extend through the native mitral valve annulus at a location adjacent the posteromedial commissure.

In certain examples, the pusher shaft 212 and the sleeve shaft 220 can be coaxial with one another, at least within the delivery sheath 204. In addition, the delivery sheath 204 can be configured to be axially movable relative to the sleeve shaft 220 and the pusher shaft 212. As described further below, a distal end of the pusher shaft 212 can be inserted into a lumen of the sleeve shaft 220 and press against the proximal end of the docking device 240 retained inside the sleeve shaft 220.

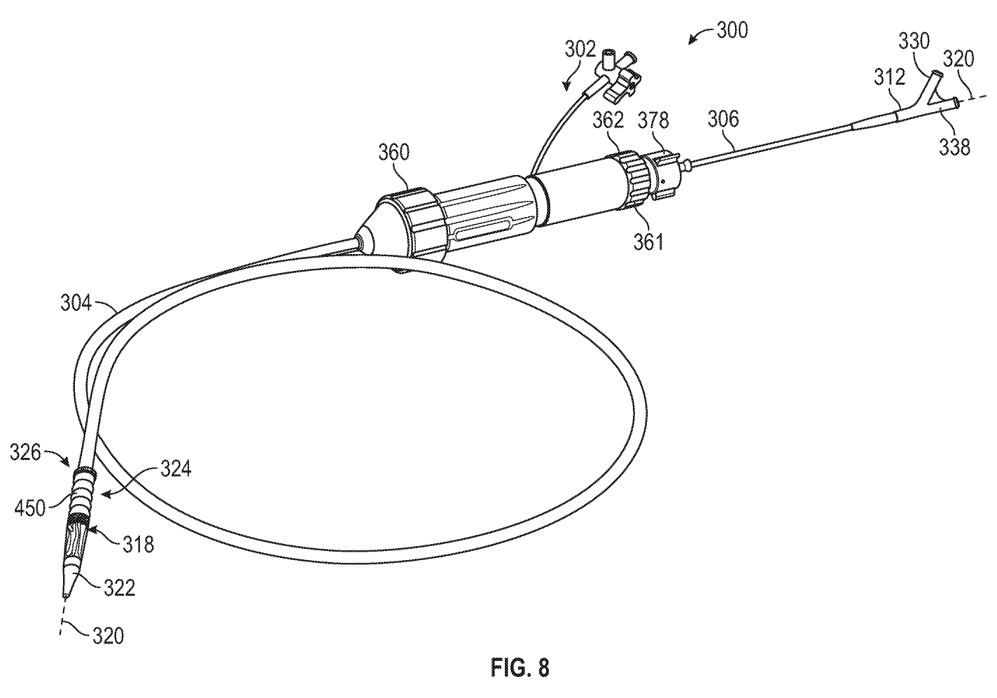

After reaching a target implantation site, the docking device 240 can be deployed from the delivery sheath 204 by manipulating the pusher shaft 212 and sleeve shaft 220 using a hub assembly 218, as described further below. For example, by pushing the pusher shaft 212 in the distal direction while holding the delivery sheath 204 in place or retracting the delivery sheath 204 in the proximal direction while holding the pusher shaft 212 in place, or pushing the pusher shaft 212 in the distal direction while simultaneously retracting the delivery sheath 204 in the proximal direction, the docking device 240 can be pushed out of a distal end 204d of the delivery sheath 204, thus changing from a delivery configuration to a deployed configuration (see FIG. 8). In certain examples, the pusher shaft 212 and the sleeve shaft 220 can be actuated independently of each other.

During delivery, the docking device 240 can be coupled to the delivery apparatus 200 via a release suture or other retrieval line comprising a string, yarn, or other material that can be configured to be tied around the docking device 240 and cut for removal that extends through the pusher shaft 212. In one specific example, the release suture can extend through the delivery apparatus 200, for example, through an inner lumen of the pusher shaft 212, to a suture lock assembly 216 of the delivery apparatus 200.

The handle assembly 202 can further include a hub assembly 218 to which the suture lock assembly 216 and a sleeve handle 224 are attached. The hub assembly 218 can be configured to independently control the pusher shaft 212 and the sleeve shaft 220 while the sleeve handle 224 can control an axial position of the sleeve shaft 220 relative to the pusher shaft 212. In this way, operation of the various components of the handle assembly 202 can actuate and control operation of the components arranged within the delivery sheath 204. In some examples, the hub assembly 218 can be coupled to the handle 206 via a connector 226.

The handle assembly 202 can further include one or more flush ports (for example, flush port 232 is shown in FIG. 6) to supply flush fluid to one or more lumens arranged within the delivery apparatus 200 (for example, annular lumens arranged between coaxial components of the delivery apparatus 200).

Further details on delivery apparatus/catheters/systems (including various examples of the handle assembly) that are configured to deliver a docking device to a target implantation site can be found in International Publication No. WO 2020/247907 and in U.S. Patent Publication Nos. 2018/0318079 and 2018/0263764, which are all incorporated by reference herein in their entireties.

FIG. 7 illustrates a docking device 240, according to one example. The docking device 240 can, for example, be used as the docking device 52 in a prosthetic valve implantation procedure, as described above with reference to FIGS. 1-4. As depicted in FIG. 7, the docking device in its deployed configuration can be configured to receive and secure a prosthetic valve therein, thereby securing the prosthetic valve at the native valve annulus.

The docking device 240 can comprise a coil 242 and a guard member 244 covering at least a portion of the coil 242. In some examples, the coil 242 can include a shape memory material (for example, nickel titanium alloy or “Nitinol”) such that the docking device 240 (and the coil 242) can move from a substantially straight configuration (or delivery configuration) when disposed within the delivery sheath 204 of the delivery apparatus 200 to a helical, deployed configuration after being removed from the delivery sheath 204.

The coil 242 has a proximal end 242p and a distal end 242d (which also respectively define the proximal and distal ends of the docking device 240). When being disposed within the delivery sheath 204 (for example, during delivery of the docking device 240 into the vasculature of a patient), a body of the coil 242 between the proximal end 242p and distal end 242d can form a generally straight delivery configuration (in other words, without any coiled or looped portions, but can be flexed or bent) so as to maintain a small radial profile when moving through a patient’s vasculature. After being removed from the delivery sheath 204 and deployed at an implant position, the coil 242 can move from the delivery configuration to the helical deployed configuration and wrap around native tissue adjacent the implant position. For example, when implanting the docking device at the location of a native valve, the coil 242 can be configured to surround native leaflets of the native valve (and the chordae tendineae that connects native leaflets to adjacent papillary muscles, if present).

The docking device 240 can be releasably coupled to the delivery apparatus 200. For example, in certain examples, the docking device 240 can be coupled to a delivery apparatus (as described above) via a release suture that can be configured to be tied to the docking device 240 and cut for removal.

As shown in FIG. 7, the coil 242 in the deployed configuration can include a leading turn 246 (or “leading coil”), a central region 248, and a stabilization turn 250 (or “stabilization coil”) around a central longitudinal axis. The central region 248 can possess one or more helical turns having substantially equal inner diameters. The leading turn 246 can extend from a distal end of the central region 248 and has a diameter greater than the diameter of the central region 248, in the illustrated example. The stabilization turn 250 can extend from a proximal end of the central region 248 and has a diameter greater than the diameter of the central region 248, in the illustrated example. In some examples, the stabilization turn 250 can be omitted from the coil 242, for example, when a retention member (such as any retention member described herein) is used to stabilize the positioning of the docking device 240 relative to the native anatomy during an implant procedure. Alternatively, the stabilization turn 250 can have a diameter that is equal, approximately equal, or less than the diameter of the central region 248 (as opposed to larger), and/or the stabilization turn can comprise less of a full turn than depicted in FIG. 8.

Further details of the docking device and its variants are described in International Publication No. WO 2022/087336, which is incorporated by reference herein in its entirety, and International Publication No. WO 2020/247907.

FIG. 8 illustrates a prosthetic heart valve delivery apparatus 300 (which can also be referred to here as an “implant catheter” and/or a “balloon catheter”) that can be used to implant an expandable prosthetic heart valve 450 (FIG. 9), according to one example. In some examples, the delivery apparatus 300 is specifically adapted for use in introducing a prosthetic heart valve into a heart. For example, the delivery apparatus 300 can be used as the prosthetic heart valve delivery apparatus 60 in a prosthetic valve implantation procedure, as described above with reference to FIG. 3A.

The delivery apparatus 300 in the illustrated example of FIG. 8 is a balloon catheter comprising a handle 302 and a steerable, outer shaft 304 extending distally from the handle 302. The delivery apparatus 300 can further comprise an intermediate shaft 306 (which also may be referred to as a balloon shaft) that extends proximally from the handle 302 and distally from the handle 302, the portion extending distally from the handle 302 also extending coaxially through the outer shaft 304. In some examples, the delivery apparatus 300 can further comprise an inner shaft extending distally from the handle 302 coaxially through the intermediate shaft 306 and the outer shaft 304 and proximally from the handle 302 coaxially through the intermediate shaft.

The outer shaft 304 and the intermediate shaft 306 can be configured to translate (e.g., move) longitudinally, along a central longitudinal axis 320 of the delivery apparatus 300, relative to one another to facilitate delivery and positioning of a prosthetic valve at an implantation site in a patient’s body.

The intermediate shaft 306 can include a proximal end portion that extends proximally from a proximal end of the handle 302, to an adaptor 312. The adaptor 312 can include a first port 338 configured to receive a guidewire therethrough and a second port 340 configured to receive fluid (e.g., inflation fluid) from a fluid source. The second port 340 can be fluidly coupled to an inner lumen of the intermediate shaft 306.

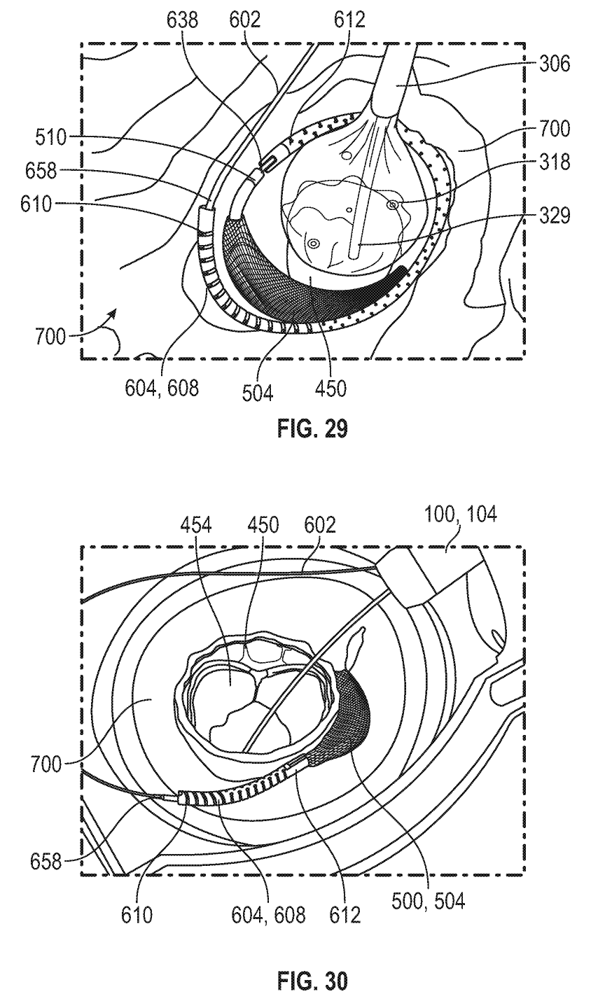

In some examples, the intermediate shaft 306 can further include a distal end portion that extends distally beyond a distal end of the outer shaft 304 when a distal end of the outer shaft 304 is positioned away from an inflatable balloon 318 of the delivery apparatus 300. A distal end portion of the inner shaft 307 (FIG. 29) can extend distally beyond the distal end portion of the intermediate shaft 306 toward or to a nose cone 322 at a distal end of the delivery apparatus 300.

In some examples, a distal end of the balloon 318 can be coupled to a distal end of the delivery apparatus 300, such as to the nose cone 322 (as shown in FIG. 8), or to an alternate component at the distal end of the delivery apparatus 300 (for example, a distal shoulder). An intermediate portion of the balloon 318 can overlay a valve mounting portion 324 of a distal end portion of the delivery apparatus 300 and a distal end portion of the balloon 318 can overly a distal shoulder of the delivery apparatus 300. As shown in FIG. 8, a prosthetic heart valve 450 can be mounted around the balloon 318, at the valve mounting portion 324 of the delivery apparatus 300, in a radially compressed state. The prosthetic heart valve 450 can be configured to be radially expanded by inflation of the balloon 318 at a native valve annulus, as described above with reference to FIG. 3A.

A balloon shoulder assembly of the delivery apparatus 300, which includes the distal shoulder, is configured to maintain the prosthetic heart valve 450 (or other medical device) at a fixed position on the balloon 318 during delivery through the patient’s vasculature.

The outer shaft 304 can include a distal tip portion 326 mounted on its distal end. In some examples, the outer shaft 304 and the intermediate shaft 306 can be translated axially relative to one another to position the distal tip portion 326 adjacent a proximal end of the valve mounting portion 324, when the prosthetic valve 450 is mounted in the radially compressed state on the valve mounting portion 324 (as shown in FIG. 8) and during delivery of the prosthetic valve to the target implantation site. As such, the distal tip portion 326 can be configured to resist movement of the prosthetic valve 450 relative to the balloon 318 proximally, in the axial direction, relative to the balloon 318, when the distal tip portion 326 is arranged adjacent a proximal side of the valve mounting portion 324.

An annular space can be defined between an outer surface of the inner shaft and an inner surface of the intermediate shaft 306 and can be configured to receive fluid from a fluid source via the second port 340 of the adaptor 312. The annular space can be fluidly coupled to a fluid passageway formed between the outer surface of the distal end portion of the inner shaft and an inner surface of the balloon 318. As such, fluid from the fluid source can flow to the fluid passageway from the annular space to inflate the balloon 318 and radially expand and deploy the prosthetic valve 450.

An inner lumen of the inner shaft can be configured to receive a guidewire therethrough, for navigating the distal end portion of the delivery apparatus 300 to the target implantation site.

The handle 302 can include a steering mechanism configured to adjust the curvature of the distal end portion of the delivery apparatus 300. In the illustrated example, for example, the handle 302 includes an adjustment member, such as the illustrated rotatable knob 360, which in turn is operatively coupled to the proximal end portion of a pull wire. The pull wire can extend distally from the handle 302 through the outer shaft 304 and has a distal end portion affixed to the outer shaft 304 at or near the distal end of the outer shaft 304. Rotating the knob 360 can increase or decrease the tension in the pull wire, thereby adjusting the curvature of the distal end portion of the delivery apparatus 300. Further details on steering or flex mechanisms for the delivery apparatus can be found in U.S. Patent No. 9,339,384.

The handle 302 can further include an adjustment mechanism 361 including an adjustment member, such as the illustrated rotatable knob 362, and an associated locking mechanism including another adjustment member, configured as a rotatable knob 378. The adjustment mechanism 361 is configured to adjust the axial position of the intermediate shaft 306 relative to the outer shaft 304 (for example, for fine positioning at the implantation site).

FIG. 9 illustrates a prosthetic heart valve 450 that can be implanted via a delivery apparatus (also referred to as an “implant catheter” and/or “balloon catheter”) in a prosthetic valve implantation procedure, such as described above with reference to FIG. 3A. For example, the delivery apparatus can be delivery apparatus 300 or delivery apparatus 60 described previously. Further details of the balloon catheter can be found, at least, in U.S. Patent No. 9,339,384, which is incorporated by reference herein.

FIG. 9 illustrates the prosthetic valve 450 in a radially expanded position. The prosthetic valve 450 can be used as the prosthetic heart valve 62 in a prosthetic valve implantation procedure, as described above with reference to FIGS. 1-4. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device (for example, docking device 240, docking device 52, docking device 500, etc.) that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in International Publication No. WO2020/247907. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

As shown in FIG. 9, the prosthetic valve 450 can include a frame 452 and a plurality of leaflets 454 situated at least partially within the frame 452. The prosthetic valve 450 can also include an outer covering 456 (which is also referred to herein as an “outer skirt”) situated about the frame 452. As shown in FIG. 9, the prosthetic valve 450 includes an inflow end 457 and an outflow end 458. The terms “inflow” and “outflow” are related to the normal direction of blood flow (for example, antegrade blood flow) through the prosthetic valve 450. For example, the leaflets 454 can allow blood flow through the valve 450 in a direction from the inflow end 457 to the outflow end 458 and prevent the reverse flow (for example, prevent flow in a direction from the outflow end 458 to the inflow end 457).

The frame 452 can be made of any of various suitable plastically-expandable materials (for example, stainless steel, etc.) or self-expanding materials (for example, Nitinol) as known in the art. When constructed of a plastically-expandable material, the frame 452 (and thus the valve 450) can be crimped to a radially compressed state on a delivery catheter and then expanded inside a patient by an inflatable balloon or equivalent expansion mechanism. When constructed of a self-expandable material, the frame 452 (and thus the valve 450) can be crimped to a radially compressed state and restrained in the compressed state by insertion into a sheath or equivalent mechanism of a delivery catheter. Once inside the body, the valve 450 can be advanced from the delivery sheath, which allows the valve 450 to expand to its functional size.

Suitable plastically-expandable materials that can be used to form the frames disclosed herein (for example, the frame 452) include, metal alloys, polymers, or combinations thereof. Example metal alloys can comprise one or more of the following: nickel, cobalt, chromium, molybdenum, titanium, or other biocompatible metal. In some examples, the frame 452 can comprise stainless steel. In some examples, the frame 452 can comprise cobalt-chromium. In some examples, the frame 452 can comprise nickel-cobalt-chromium. In some examples, the frame 452 comprises a nickel-cobalt-chromium-molybdenum alloy, such as MP35N™ (tradename of SPS Technologies), which is equivalent to UNS R30035 (covered by ASTM F562-2). MP35N™/UNS R30035 comprises 35% nickel, 35% cobalt, 20% chromium, and 10% molybdenum, by weight.

The outer skirt 456 can be wholly or partly formed of any suitable biological material, synthetic material (for example, any of various polymers), or combinations thereof. In some examples, the outer skirt 456 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the outer skirt 456 can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the outer skirt 456 can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the outer skirt 456 can comprise a sponge material or foam, such as polyurethane foam. In some examples, the outer skirt 456 can comprise natural tissue, such as pericardium (for example, bovine pericardium, porcine pericardium, equine pericardium, or pericardium from other sources).

Further details of the prosthetic heart valve and its variants are described in U.S. Patent No. 11,185,406, which is incorporated by reference herein in its entirety.

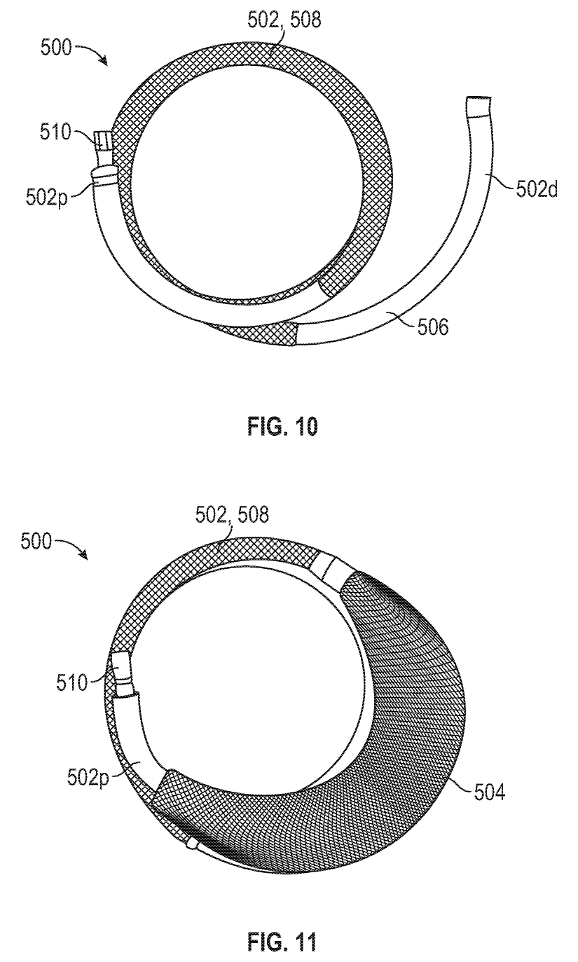

FIGS. 10-30 illustrate another example of a docking device 500 and a delivery system configured to implant a docking device and a prosthetic heart valve, the delivery system comprising a stabilizing device 600 (FIG. 13). Docking device 500 and stabilizing device 600 are configured to help retain the positioning of the docking device 500 relative to the native tissue during the transient stage of the implantation procedure (e.g., following docking device implantation and prior to prosthetic valve implantation). Docking device 500 and stabilizing device 600 can, for example, be used in a prosthetic valve implantation procedure, such as described above with reference to FIGS. 1-4.

Referring to FIGS. 10-12, docking device 500 can be similar to docking device 240 described previously, except that docking device 500 does not include the stabilizing turn 250. That is, docking device 500 can comprise a coil 502 and an optional guard member 504 (FIG. 11) covering at least a portion of the coil 502. In some examples, the coil 502 can include a shape memory material (for example, nickel titanium alloy or “Nitinol”) such that the docking device 500 (and the coil 502) can move from a substantially straight configuration (or delivery configuration) when disposed within a delivery sheath/shaft of a delivery apparatus (e.g., within shaft 220 of the dock delivery apparatus 200) to a helical, deployed configuration after being removed from the delivery sheath.

The coil 502 has a proximal end 502p and a distal end 502d (which also respectively define the proximal and distal ends of the docking device 500). When disposed within the dock delivery apparatus (for example, during delivery of the docking device 500 into the vasculature of a patient), a body of the coil 502 between the proximal end 502p and distal end 502d can form a generally straight delivery configuration (in other words, without any coiled or looped portions, but can be flexed or bent) so as to maintain a small radial profile when moving through a patient’s vasculature. After being removed from the dock delivery apparatus and deployed at an implant position, the coil 502 can move from the delivery configuration to the helical deployed configuration and wrap around native tissue adjacent the implant position. For example, when implanting the docking device 500 at the location of a native valve, the coil 502 can be configured to surround native leaflets of the native valve (and the chordae tendineae that connects native leaflets to adjacent papillary muscles, if present).

As shown in FIG. 10, the coil 502 in the deployed configuration can include a leading turn 506 (or “leading coil”) and a central region 508 around a central longitudinal axis (extending into/out of the page in the orientation shown in FIG. 10). The central region 508 can possess one or more helical turns having substantially equal inner diameters. The leading turn 506 can extend from a distal end of the central region 508 and has a diameter greater than the diameter of the central region 508, in the illustrated example. Unlike docking device 240, the docking device 500 does not include a stabilization turn.

Referring to FIG. 11, as mentioned previously, the docking device 500 can optionally comprise a guard member 504. The guard member 504 can be configured to prevent or mitigate paravalvular leakage between the annulus of the native mitral valve and a prosthetic heart valve (such as the prosthetic heart valve 450) positioned in the docking device 500. Further details of docking devices and guard members can be found, at least, in PCT Publication WO 2020/247907, which is incorporated by reference herein in its entirety.

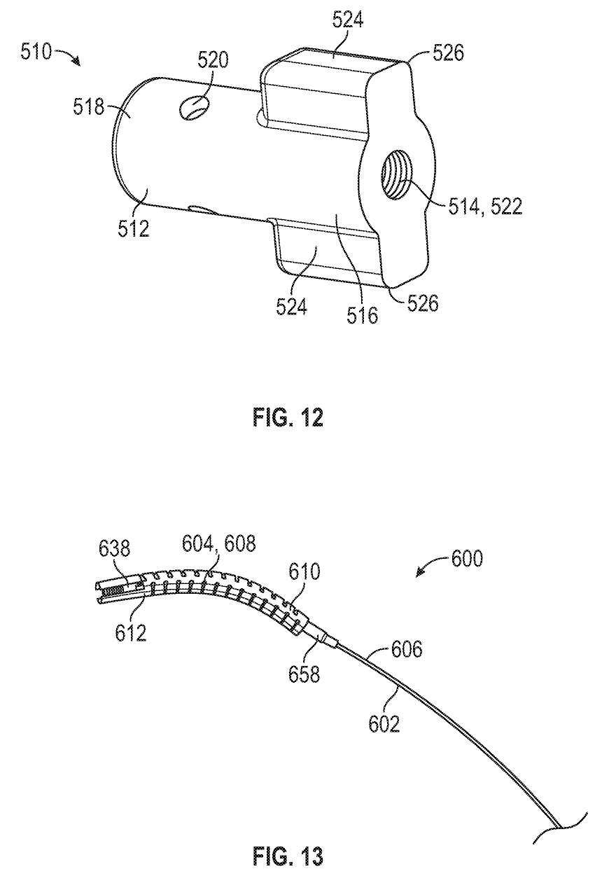

As shown in FIGS. 10-12, the docking device 500 can comprise a proximal nut 510 used to couple to the docking device 500 to a stabilizing device 600 (FIG. 13). Referring to FIG. 12, the proximal nut 510 can include a main body portion 512 that defines an inner bore 514. The main body 512 can comprise a proximal end portion 516 and a distal end portion 518. The distal end portion 518 can be coupled to the proximal end 502p (FIG. 10) of the docking device 500. For example, the distal end portion 518 can be welded, sewn on, or crimped to the docking device 500. In some examples, the distal end portion 518 can comprise one or more apertures/openings 520 extending through a sidewall of the main body 512. The apertures/openings 520 can be used to facilitate attachment of the distal end portion 518 to the docking device 500, for example, by facilitating welding. The proximal end portion of the inner bore 514 includes an engagement portion 522 (e.g., a threaded portion in the illustrated example) configured to mate with a coupling member 638 of the hypotube member 604 (FIG. 13) of the stabilization device 600, as described in more detail below.

Referring still to FIG. 12, the main body 512 can comprise one or more projections or wing members 524 that extend radially from an outer surface of the main body 512. The wing members 524 can engage corresponding cutouts 622 of the hypotube member 604 (FIG. 13) to prevent or mitigate rotation of the hypotube member 604 relative to the docking device 500 when the docking device 500 is coupled to a stabilization device 600. In the illustrated example, the proximal nut 510 includes two diametrically opposed wing members 524. In some examples, the proximal nut 510 can include a greater or fewer number of wing members, for example, one, three, four, or five wing members. As shown in FIG. 12, in some examples, the wing members 524 can have an elongated, rectangular shape including rounded corner edges 526. In some examples, the corner edges 526 can be chamfered. In some examples, the wing members 524 can have any of various other shapes.

As mentioned above, the docking device 500 can be implanted using a delivery system including the guide sheath 100, the dock delivery device 200, a prosthetic valve delivery apparatus 300, all described previously, and further including a stabilizing device 600, shown in FIG. 13. Docking device 500, guide catheter 100, dock delivery apparatus 200, and prosthetic valve delivery apparatus 300 can, for example, be used as the docking device 52, guide catheter 30, dock delivery apparatus 50, and prosthetic valve delivery apparatus 60 in a prosthetic valve implantation procedure, such as described above with reference to FIGS. 1-4. In some examples, the procedure can further comprise stabilizing device 600, as described in more detail below.

Referring to FIGS. 13-26, as mentioned, the delivery system can further comprise a stabilizing device 600. The stabilizing device 600 can be coupled to the docking device 500 to help maintain the positioning of the docking device 500 at a desired implantation location. In some examples, this can include retaining the docking device 500 in a desired position and/or orientation and/or improving the stability of the docking device 500. The stabilizing device 600 can comprise a wire 602 and a hypotube member 604. As discussed further below, the wire 602 and hypotube member 604 can remain coupled to the docking device 500 during the transient stage of the implantation procedure (e.g., after removal of the dock delivery apparatus and before implantation of a prosthetic valve within the docking device 500) in order to retain the docking device 500 relative to the native anatomy.

As mentioned, the stabilizing device 600 can comprise a wire 602 and a hypotube member 604. Referring to FIG. 13, the wire 602 can have a distal end portion 606 coupled to the hypotube member 604, and a proximal end portion extending distally from the handle 206 of the dock delivery device 200. Once the docking device 500 has been implanted, the proximal end of the wire 602 can be released from the dock delivery device 200, leaving the stabilization device within the guide sheath 100 after retraction of the dock delivery device 200. The wire 602 is narrow enough to allow adequate room for insertion of the prosthetic valve delivery apparatus 300 through the guide catheter 100 adjacent the wire 602.

Referring to FIG. 14, the hypotube member 604 can have a main body 608 including a proximal end portion 610 configured to be coupled to the wire 602, and a distal end portion 612 configured to releasably couple the docking device 500. The main body 608 can have a length between about 20 mm to about 250 mm, for example, from about 25 mm to about 50 mm, from about 25 mm to about 80 mm, from about 25 mm to about 100 mm, from about 25 to about 150 mm, from about 20 mm to about 175 mm.

The main body 608 comprises a plurality of cuts/openings/slots 614 arranged to provide desired material qualities. In the illustrated example, the proximal end portion 610 can comprise a spiral cut 616 and the distal end portion 612 can comprise a plurality of slots 618 separated from one another by circumferentially-extending members or ribs 620. Such a configuration provides flexibility in plane with the slots at the proximal end portion 610 and allows flexibility in plane with the slots but stiffness in torque at the distal end portion 612, thereby resisting rotation of the docking device relative to the hypotube member 604, stabilizing the docking device 500 and ensuring that it remains positioned at the selected location. The proximal end portion 610 advantageously allows for delivery system maneuverability within the native anatomy (e.g., the native atrium). The distal end portion 612 is configured to advantageously stabilize the docking device 500 and maintain the height of the docking device 500 within the native anatomy without requiring active tension on the docking device 500.

In other examples, the spiral cut 616 can extend the entire length of the main body 608, or the plurality of slots 618 and circumferentially-extending ribs 620 can extend the entire length of the main body 608. Such an example is shown in FIG. 18. The length of the spiral cut portion and/or the slotted portion can be varied as necessary to provide a desired flexibility and resistance to torque. In some examples, the spiral cut portion and/or slotted portion can be longer or shorter than what is depicted in the figures.

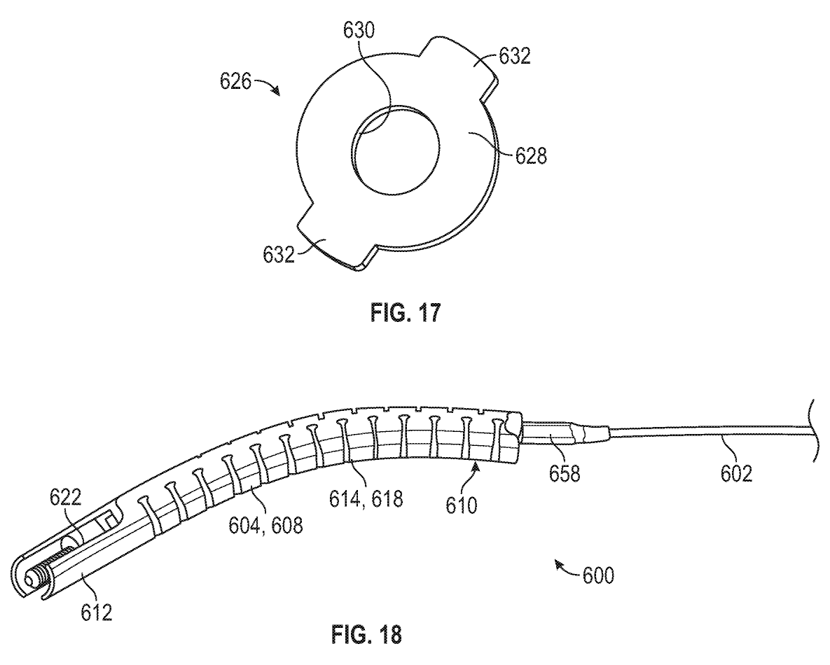

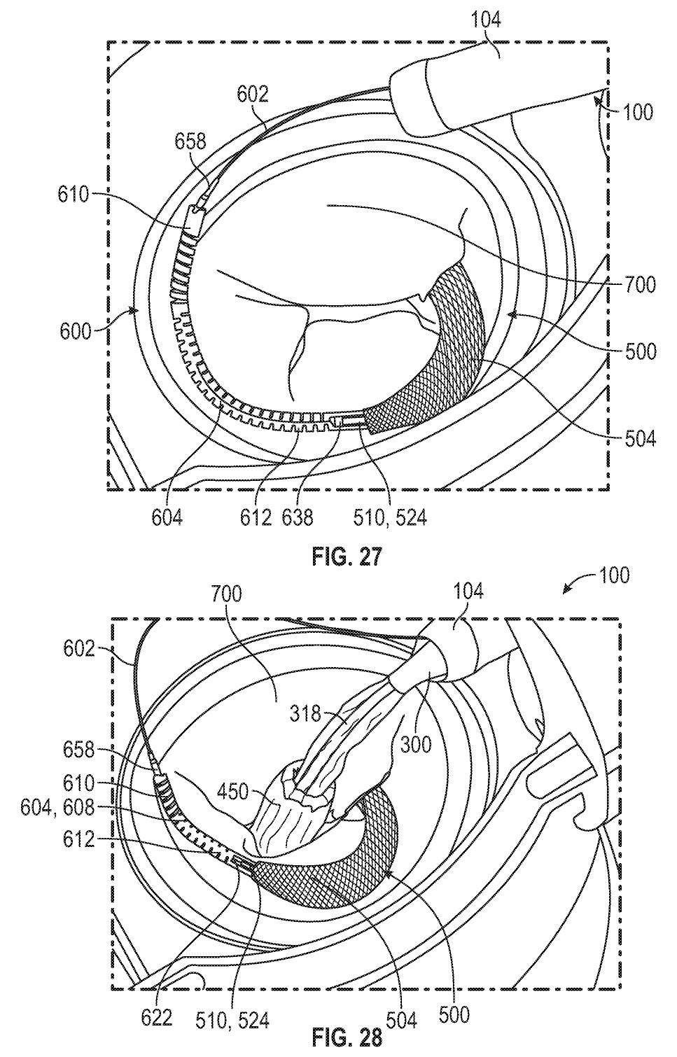

The distal end portion 612 can further comprise one or more distal cutouts 622 configured to engage the proximal nut 510 on the docking device 500. For example, each wing member 524 can be disposed within a distal cutout 622, as shown, e.g., in FIGS. 27-28. Referring again to FIG. 12, the proximal end portion 610 can comprise one or more proximal cutouts 624. The hypotube member 604 can further comprise one or more window members 626 (FIG. 17) configured to be welded or otherwise coupled to the proximal and/or distal end portions 610, 612 of the hypotube member 604. The window members 626 are configured to mitigate axial movement of the wire 602 within the hypotube member 604.

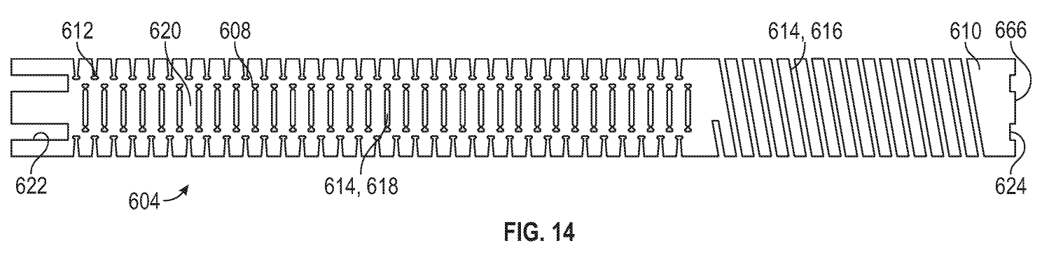

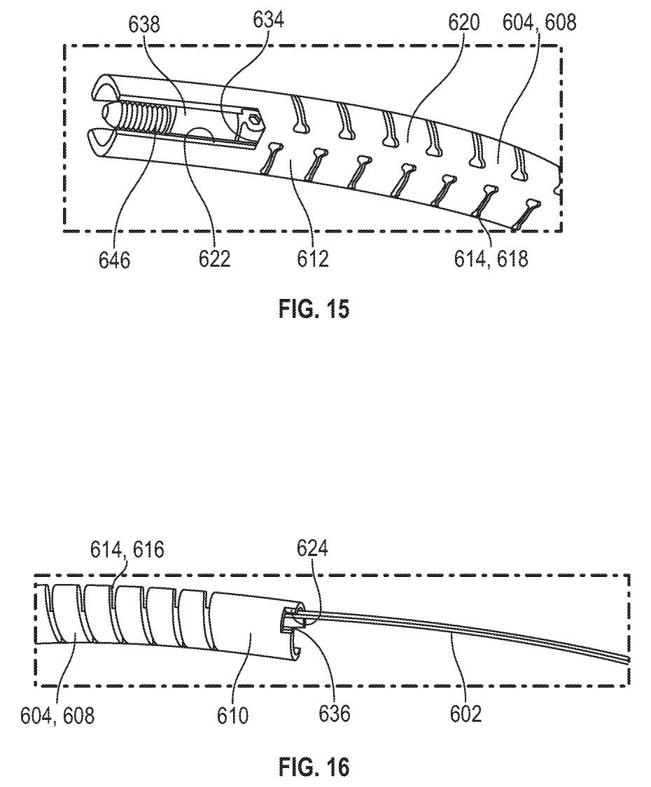

Referring to FIG. 17, each window member 626 can comprise an annular main body 628 including a central opening 630 and one or more projections 632. In the illustrated example, the projections 632 are disposed diametrically opposite one another on an outer circumference of the annular main body 628. In some examples, a window member 626 is coupled to the distal end portion 612 of the hypotube member 604 at a proximal edge 634 (FIG. 15) of the distal cutouts 622. In some examples, a window member 626 is coupled to the proximal end portion 610 of the hypotube member 604 at a distal edge 636 (FIG. 16) of the proximal cutouts 624.

In some examples, the hypotube member 604 can comprise a covering, such as a polymeric or textile covering, disposed over the axial length of the hypotube member 604. The covering can advantageously provide a smooth/cushioned surface against the native anatomy and enable additional stability of the docking device 500 at the selected implantation location, while still allowing flexibility of the stabilization device within the guide sheath 100 and at the selected implantation site. In some examples, the hypotube covering can comprise one or more of FEP tubing, a block copolymer such as Pebax, braided textiles such as PET. In some examples, the covering can comprise a hydrophilic coating. In some examples, the coating or covering of the hypotube member 604 can be configured to add friction, which can advantageously maintain stability of the device against the native anatomy.

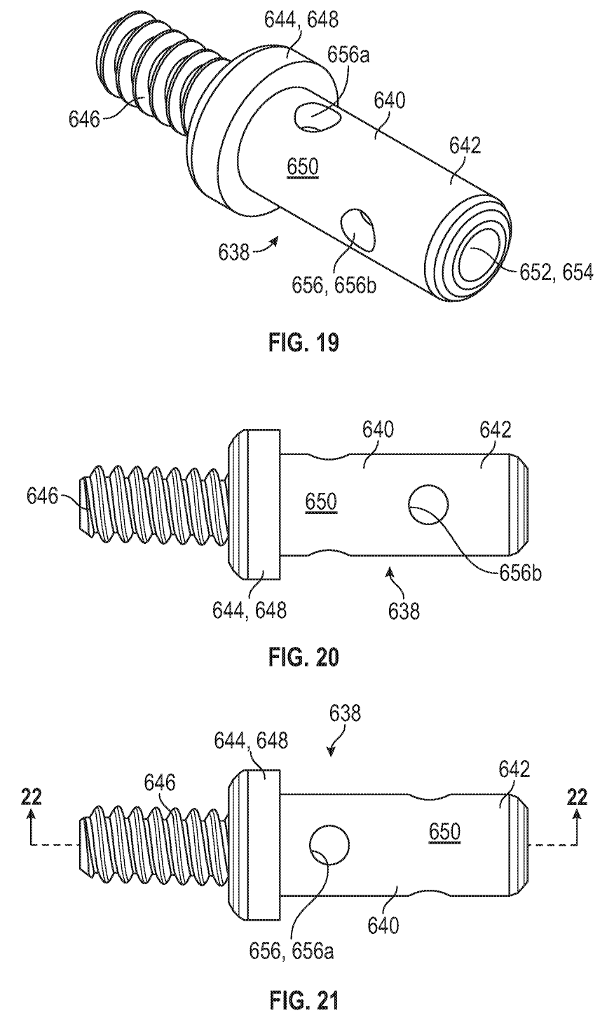

As shown in FIG. 15, the distal end portion 612 of the hypotube member 604 can be coupled to a coupling member 638. The coupling member 638 can be configured to releasably couple the docking device 500 and also to couple the hypotube member 604 to the wire 602 (which extends through the hypotube member 604 to the distal end thereof). Referring to FIGS. 19-22, the coupling member 638 can have a main body 640 including a proximal end portion 642 and a distal end portion 644. The distal end portion 644 can comprise an engagement portion 646 extending distally therefrom and configured to releasably couple a corresponding engagement portion 522 on the docking device 500 (e.g., the threaded inner bore 514 of the proximal nut 510). In the illustrated example, the engagement portion 646 is a threaded member extending distally from the main body 640 and configured to interface with the threaded inner bore 514 of the proximal nut 510. In other examples, the engagement portion 646 can interface/engage the proximal end portion of the docking device 500 using any of various other mechanisms, e.g., a snap-fit or other mechanical means. In some examples, the distal end portion 644 can comprise an annular projection 648 extending from an outer surface 650 of the main body 640. The annular projection 648 can help mitigate axial movement of the coupling member 638 with respect to the hypotube member 604.

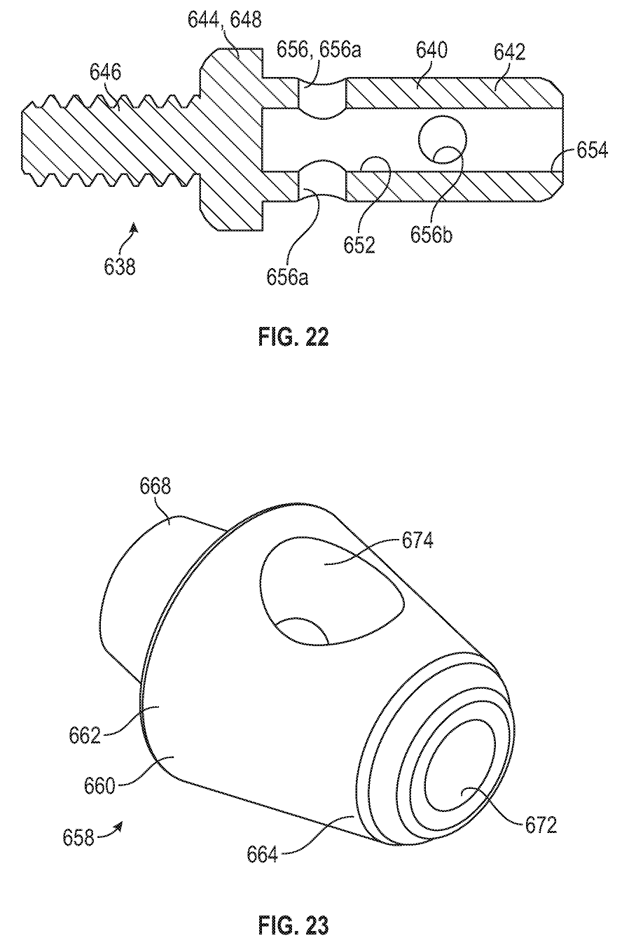

As shown in FIG. 22, the main body 640 can comprise an inner bore 652 extending from a proximal opening 654 at the proximal end portion 642 to the annular projection 648. The main body 640 can further comprise one or more openings/apertures 656 that extend through a side wall of the main body 640. In the illustrated embodiment, the main body 640 can comprise four openings 656, which are arranged in two diametrically opposed pairs, with the first pair 656a disposed adjacent the annular projection 648 and the second pair 656b disposed adjacent the proximal opening 654. The first and second pairs of openings 656a, 656b are disposed such that they are circumferentially offset from one another. The openings 656 can be used to couple the wire 602 to the coupling member 638 (and thus to the hypotube member 604), e.g., using a laser weld or other coupling technique. For example, the distal end portion 606 of the wire 602 can be inserted into the coupling member’s inner bore 652 and, thus positioned, can be welded to the coupling member 638.

As shown in FIG. 18, the proximal end portion 610 of the hypotube member 604 can be coupled to a proximal stopper 658. The proximal stopper 658 can be configured to help mitigate movement of the wire 602 within the hypotube member 604. FIGS. 23-26 illustrate various views of the proximal stopper 658. The proximal stopper 658 can comprise a main body 660 having a distal end portion 662 and a proximal end portion 664. The main body 660 can have a generally frustoconical shape tapering from a first width W₁ at the proximal end portion 664 to a second, larger width W₂ at the distal end portion 662. The tapered shape of the proximal stopper 658 advantageously provides a ramped surface for an outer shaft advanced over the proximal stopper 658 (such as guide shaft 104), thereby preventing or mitigating the outer shaft from catching on the proximal edge 666 (FIG. 14) of the hypotube member 604.

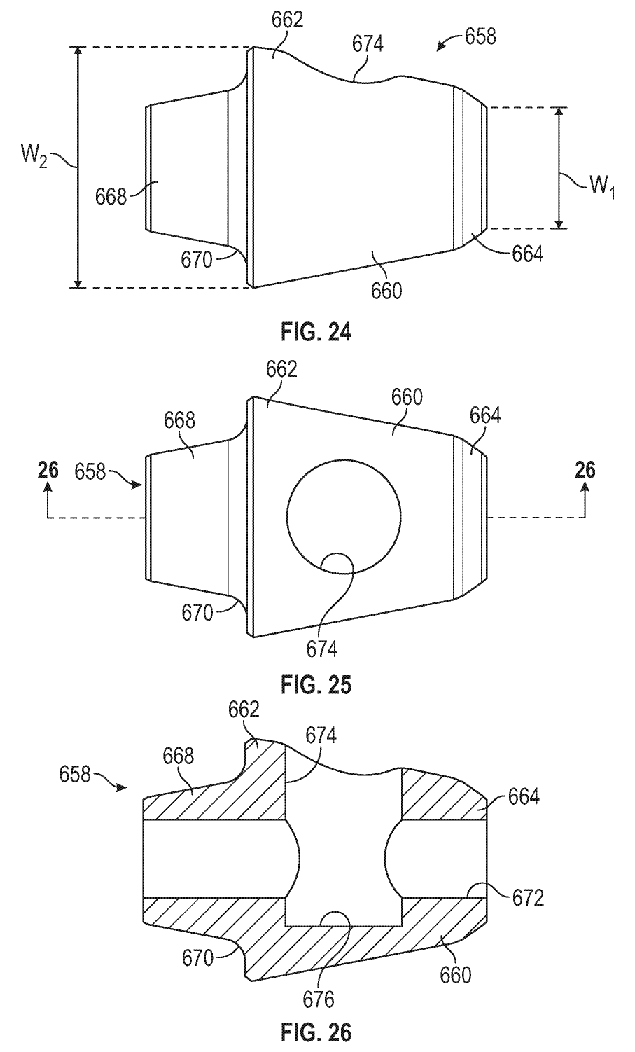

The proximal stopper 658 can further comprise an annular projection 668 extending from the distal end portion 662. The annular projection 668 can be disposed within the proximal end portion 610 of the hypotube 604. As can be seen in FIGS. 24-26, the location where the annular projection 668 extends from the main body 660 can comprise a curved or chamfered portion 670. As shown in FIG. 26, the main body 660 defines an inner bore 672 extending axially through the main body 660. The main body 660 further comprises an opening/aperture 674 extending through a side wall of the main body 660. The opening 674 can extend to the inner bore 672 and be contiguous therewith. As can be seen in FIG. 26, the inner bore 672 can comprise a recess 676 diametrically opposite the opening 674 on an inner surface of the main body 660 defined by the inner bore 672.

As mentioned, the wire 602 can extend into the hypotube member 604 and can be coupled to the distal end portion 612 of the hypotube member 604 via the coupling member 638. The wire 602 can extend through the proximal stopper 658 and through the one or more window members 626. In some examples, the wire 602 can comprise Nitinol.

As shown in FIG. 27, the stabilizing device 600 can extend through the elongated shaft 104 of the guide catheter 100. In some examples, the stabilizing device 600 can extend through the main lumen of the elongated shaft 34 or 104. In some examples, the stabilizing device 600 can extend through a separate lumen of the elongated shaft 104.

The stabilizing device 600 can be used as part of the delivery system to implant the docking device 500 and a prosthetic valve (e.g., prosthetic valve 450 described previously) in a subject’s native mitral valve 700 in the following exemplary manner.

A user first uses a guide catheter, such as guide catheter 30 or 100, to create a pathway to a patient’s native heart valve, as shown in FIG. 1 and described with reference thereto. The pathway created can be, for example, a transseptal pathway. As described previously with reference to FIG. 2A-2B, the docking device 500 can then be deployed/implanted at the native mitral valve 700 using a dock delivery apparatus, such as dock delivery apparatus 50 or 200, to advance the docking device 500 through the shaft 34/104 of the guide catheter 30/100. The stabilizing device 600 can be coupled to the docking device 500 and can be advanced through the shaft 34/104 in connection with the docking device 500 as the docking device 500 is advanced to the selected implantation site. During the implantation procedure, the stabilization device 600 can be coupled to the dock delivery apparatus 50/200. Once the docking device 500 is deployed/implanted at the implantation site, the user may disconnect the dock delivery apparatus 50/200 and retract it through the elongated shaft 104 and out of the patient, leaving the stabilization device 600 connected to the docking device 500.

FIGS. 27-30 proceed with reference to guide catheter 100, dock delivery apparatus 200, prosthetic valve delivery apparatus 300, and prosthetic valve 450, however, it should be noted that guide catheter 30, dock delivery apparatus 50, prosthetic valve delivery apparatus 60, and prosthetic heart valve 62 could alternatively be used. As shown in FIG. 27, the docking device 500 is thus deployed and implanted at the selected implantation site (in the present example, the native mitral valve 700) with the stabilization device 600 connected thereto. The docking device 500 can wrap around the leaflets of the native mitral valve 700 (within the left ventricle) as discussed previously with reference to FIG. 2B. The wire 602 of the stabilization device 600 can extend into the elongated shaft 104 of the guide catheter 100. In some examples, at this point in the implantation procedure, the proximal end portion of the wire 602 has been uncoupled from the dock delivery apparatus 200. The attachment of the stabilization device 600 to the docking device 500 can advantageously maintain the docking device 500 at the selected implantation location after disconnection from the dock delivery apparatus 200 and before implantation of a prosthetic heart valve (such as prosthetic heart valve 450).

The user can then deliver and/or implant the prosthetic heart valve 450 within the docking device 500 using the prosthetic valve delivery apparatus 300, while the docking device is stabilized using the stabilization device 600. Referring to FIG. 28, the distal end portion of the prosthetic valve delivery apparatus 300 is advanced through the guide catheter 100 until the radially compressed prosthetic heart valve 450 (which is disposed around inflatable balloon 318) is positioned within the docking device 500 and the native mitral valve 700.

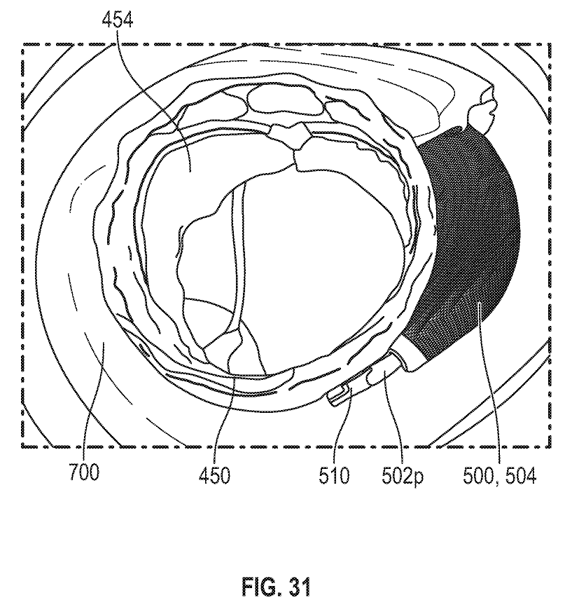

Once the radially compressed prosthetic heart valve 450 is appropriately positioned within the docking device 500, as shown in FIG. 28, the user can manipulate one or more actuation mechanisms of the handle 302 of the prosthetic valve delivery apparatus 300 to actuate the expansion mechanism (e.g., the balloon 318), as shown in FIG. 29, thereby radially expanding the prosthetic heart valve 450 within the docking device 500, as shown in FIG. 30. The stabilization device 600 can then be disconnected/uncoupled from the docking device 500, for example, by rotating the coupling member 638 such that it is unthreaded from the proximal nut 510 of the docking device 500. The prosthetic valve delivery apparatus 300 and the stabilization device 600 can then be removed from the patient. The guide catheter 100 can then be removed from the patient, as shown in FIG. 31.

Although described in the context of a mitral valve replacement procedure, it should be appreciated that the same and/or similar procedure may be utilized to replace other heart valves (for example, tricuspid, pulmonary, and/or aortic valves). Further, the same and/or similar delivery apparatuses (for example, docking device delivery apparatus 50/200, prosthetic valve delivery apparatus 60/300, and/or guide catheter 30/100), stabilizing devices 600, docking devices (for example, docking device 52/240/500), replacement heart valves (for example, prosthetic heart valve 62/450), and/or components thereof may be utilized for replacing these other heart valves.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An assembly, comprising: a docking device comprising a coil extending along a central axis and including a central region including a plurality of turns, and a leading turn extending from a distal end portion of the central region; and a stabilization device releasably coupled to a proximal end portion of the docking device, the stabilization device comprising a wire and a hypotube member having a first portion comprising a spiral cut and a second portion comprising a plurality of slots; wherein the stabilization device is configured to maintain the docking device at a selected implantation location during an implantation procedure.

Example 2. The assembly of any example herein, particularly example 1, wherein the hypotube member comprises a proximal end portion and a distal end portion, and wherein the distal end portion comprises a coupling member having an engagement portion releasably couplable to a corresponding engagement portion of the docking device.

Example 3. The assembly of any example herein, particularly example 2, wherein the engagement portion is a threaded member extending distally from the distal end portion of the coupling member and the corresponding engagement portion is a correspondingly threaded inner bore disposed at the proximal end portion of the docking device.

Example 4. The assembly of any example herein, particularly example 3, wherein the correspondingly threaded inner bore is defined by a proximal nut coupled to a proximal end portion of the docking device.

Example 5. The assembly of any example herein, particularly example 4, wherein the proximal nut further comprises one or more wing members extending radially from a main body of the proximal nut, the one or more wing members configured to engage one or more cutouts on the distal end portion of the hypotube member to prevent rotation of the docking device relative to the hypotube member.

Example 6. The assembly of any example herein, particularly any one of examples 1-5, wherein the hypotube member further comprises a proximal stopper coupled to a proximal end portion of the main body, the proximal stopper comprising a tapered main body that tapers from a first width at a proximal end portion to a second, larger width at a distal end portion.

Example 7. A delivery system, comprising: a docking device comprising a coil extending along a central axis and including a central region including a plurality of turns, and a leading turn extending from a distal end portion of the central region; and a stabilization device comprising a wire and a hypotube member, the hypotube member comprising: a main body, and a coupling member coupled to a distal end portion of the main body, the coupling member configured to releasably couple a proximal end portion of the docking device, wherein the stabilization device is configured to maintain the docking device at a selected implantation location during an implantation procedure.

Example 8. The system of any example herein, particularly example 7, wherein the hypotube member further comprises a proximal stopper coupled to a proximal end portion of the main body, the proximal stopper comprising a main body tapering from a first width at a proximal end portion to a second, larger width at a distal end portion.

Example 9. The system of any example herein, particularly any one of examples 7-8, wherein the coupling member comprises an engagement portion configured to releasably couple a corresponding engagement portion of the docking device.

Example 10. The system of any example herein, particularly example 9, wherein the engagement portion is a threaded member extending distally from the main body of the hypotube member.

Example 11. The system of any example herein, particularly any one of examples 7-10, wherein the hypotube member comprises a first portion comprising a spiral cut and a second portion comprising a plurality of slots separated by a plurality of circumferentially extending ribs.

Example 12. The system of any example herein, particularly any one of examples 7-11, wherein the docking device further comprises a proximal nut coupled to the proximal end portion, the proximal nut comprising the corresponding engagement portion and further comprising one or more wing members configured to interface with cutouts in the distal end portion of the hypotube member to prevent torque.

Example 13. The system of any example herein, particularly example 12, wherein the corresponding engagement portion is a threaded inner bore extending into a main body of the proximal nut.

Example 14. The system of any example herein, particularly any one of examples 7-13, further comprising a prosthetic heart valve configured to be implanted within the docking device, the prosthetic heart valve comprising a radially expandable and compressible frame and a leaflet structure positioned within the frame and secured thereto.

Example 15. A method, comprising: implanting a docking device at a selected implantation location in a native annulus of a subject’s heart using a dock delivery apparatus, the docking device coupled to a stabilization device comprising a wire and a hypotube member; removing the dock delivery apparatus while maintaining the docking device at the selected implantation location using the stabilization device; using a prosthetic heart valve delivery apparatus, advancing a prosthetic heart valve mounted to an inflatable balloon of the delivery apparatus to the selected implantation location; positioning the prosthetic heart valve and inflatable balloon within the docking device; inflating the balloon to radially expand the prosthetic heart valve while maintaining the docking device at the selected implantation location using the stabilization device; and uncoupling the stabilization device from the docking device; and removing the stabilization device and the prosthetic heart valve delivery apparatus from the subject.

Example 16. The method of any example herein, particularly example 15, further comprising: prior to implanting the docking device at the selected location, advancing a guide catheter shaft to the selected implantation location.

Example 17. The method of any example herein, particularly example 16, further comprising: advancing the dock delivery apparatus, docking device, and stabilization device through the guide catheter shaft to the selected implantation location.

Example 18. A system, comprising: a guide catheter including a handle and a guide catheter shaft extending distally from the handle; a docking device comprising a coil extending along a central axis and including a central region including a plurality of turns, and a leading turn extending from a distal end portion of the central region; and a stabilization device extending distally through the guide catheter shaft and releasably coupled to the docking device, the stabilization device comprising a wire and a hypotube member; wherein the stabilization device is configured to retain the docking device at a selected implantation location during an implantation procedure wherein a prosthetic heart valve is implanted within the docking device.

Example 19. The system of any example herein, particularly example 18, wherein the hypotube member comprises a first end portion including a spiral cut configured to allow flexibility of the first end portion in all directions and a second end portion including a plurality of slots configured to allow flexibility in plane but resist rotation of the hypotube member relative to the docking device.

Example 20. The system of any example herein, particularly any one of examples 18-19, wherein the docking device comprises a nut coupled to a proximal end thereof, the nut comprising a threaded inner bore releasably coupled to a threaded member of the hypotube member.

Example 21. The system according to any example herein, wherein any one component of the system has been sterilized.

Example 22. A method of sterilizing any one of the components of the system described herein.

The features described herein with regard to any example can be combined with other features described in any one or more of the other examples, unless otherwise stated. For example, any one or more of the features of one sealing member can be combined with any one or more features of another sealing member or another prosthetic valve. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. An assembly comprising:
a docking device comprising a coil extending along a central axis and including a central region including a plurality of turns, and a leading turn extending from a distal end portion of the central region; and
a stabilization device releasably coupled to a proximal end portion of the docking device, the stabilization device comprising a wire and a hypotube member having a first portion comprising a spiral cut and a second portion comprising a plurality of slots;
wherein the stabilization device is configured to maintain the docking device at a selected implantation location during an implantation procedure.

2. The assembly of claim 1, wherein the hypotube member comprises a proximal end portion and a distal end portion, and wherein the distal end portion comprises a coupling member having an engagement portion releasably couplable to a corresponding engagement portion of the docking device.

3. The assembly of claim 2, wherein the engagement portion is a threaded member extending distally from the distal end portion of the coupling member and the corresponding engagement portion is a correspondingly threaded inner bore disposed at the proximal end portion of the docking device.

4. The assembly of claim 3, wherein the correspondingly threaded inner bore is defined by a proximal nut coupled to a proximal end portion of the docking device.

5. The assembly of claim 4, wherein the proximal nut further comprises one or more wing members extending radially from a main body of the proximal nut, the one or more wing members configured to engage one or more cutouts on the distal end portion of the hypotube member to prevent torque.

6. The assembly of claim 1, wherein the hypotube member further comprises a proximal stopper coupled to a proximal end portion of the main body, the proximal stopper comprising a tapered main body that tapers from a first width at a proximal end portion to a second, larger width at a distal end portion.

7. A delivery system comprising:
a docking device comprising a coil extending along a central axis and including a central region including a plurality of turns, and a leading turn extending from a distal end portion of the central region; and
a stabilization device comprising a wire and a hypotube member, the hypotube member comprising:
a main body, and
a coupling member coupled to a distal end portion of the main body, the coupling member configured to releasably couple a proximal end portion of the docking device,
wherein the stabilization device is configured to maintain the docking device at a selected implantation location during an implantation procedure.

8. The system of claim 7, wherein the hypotube member further comprises a proximal stopper coupled to a proximal end portion of the main body, the proximal stopper comprising a main body tapering from a first width at a proximal end portion to a second, larger width at a distal end portion.

9. The system of claim 7, wherein the coupling member comprises an engagement portion configured to releasably couple a corresponding engagement portion of the docking device.

10. The system of claim 9, wherein the engagement portion is a threaded member extending distally from the main body of the hypotube member.

11. The system of claim 7, wherein the hypotube member comprises a first portion comprising a spiral cut and a second portion comprising a plurality of slots separated by a plurality of circumferentially extending ribs.

12. The system of claim 7, wherein the docking device further comprises a proximal nut coupled to the proximal end portion, the proximal nut comprising the corresponding engagement portion and further comprising one or more wing members configured to interface with cutouts in the distal end portion of the hypotube member to resist rotation of the docking device relative to the hypotube member.

13. The system of claim 12, wherein the corresponding engagement portion is a threaded inner bore extending into a main body of the proximal nut.

14. The system of claim 7, further comprising a prosthetic heart valve configured to be implanted within the docking device, the prosthetic heart valve comprising a radially expandable and compressible frame and a leaflet structure positioned within the frame and secured thereto.

15. A system, comprising:
a guide catheter including a handle and a guide catheter shaft extending distally from the handle;
a docking device comprising a coil extending along a central axis and including a central region including a plurality of turns, and a leading turn extending from a distal end portion of the central region; and
a stabilization device extending distally through the guide catheter shaft and releasably coupled to the docking device, the stabilization device comprising a wire and a hypotube member;
wherein the stabilization device is configured to retain the docking device at a selected implantation location during an implantation procedure wherein a prosthetic heart valve is implanted within the docking device.

16. The system of claim 15, wherein the hypotube member comprises a first end portion including a spiral cut configured to allow flexibility of the first end portion in all directions and a second end portion including a plurality of slots configured to allow flexibility in plane but resist rotation of the hypotube member relative to the docking device.

17. The system of claim 15, wherein the docking device comprises a nut coupled to a proximal end thereof, the nut comprising a threaded inner bore releasably coupled to a threaded member of the hypotube member.

18. The system of claim 15, further comprising a prosthetic heart valve configured to be implanted within the docking device, the prosthetic heart valve comprising a radially expandable and compressible frame and a leaflet structure positioned within the frame and secured thereto.

19. The system of claim 16, further comprising a prosthetic heart valve configured to be implanted within the docking device, the prosthetic heart valve comprising a radially expandable and compressible frame and a leaflet structure positioned within the frame and secured thereto.

20. The system of claim 17, further comprising a prosthetic heart valve configured to be implanted within the docking device, the prosthetic heart valve comprising a radially expandable and compressible frame and a leaflet structure positioned within the frame and secured thereto.
